(19) United States
(12) Patent Application Publication
(10) Pub. No.: US 2025/0090081 A1
(43) Pub. Date: Mar. 20, 2025

(54) BALANCE AND GAIT ASSISTANCE SYSTEMS

(71) Applicants: Ruth Kaluza Eichholtz, Wilsonville, OR (US); Charles Kaluza, Wilsonville, OR (US)

(72) Inventors: Ruth Kaluza Eichholtz, Wilsonville, OR (US); Charles Kaluza, Wilsonville, OR (US)

(21) Appl. No.: 18/885,301

(22) Filed: Sep. 13, 2024

Related U.S. Application Data
(60) Provisional application No. 63/538,714, filed on Sep. 15, 2023.

(51) Int. Cl. A61B 5/00; A61B 5/103

(52) U.S. Cl. CPC A61B 5/4023; A61B 5/0022; A61B 5/1038; A61B 5/112; A61B 5/6807; A61B 5/7415

(57) ABSTRACT

Balance and gait assistance systems including a sensor, a computing device, and an auditory device. The sensor is disposed proximate a foot of a user and configured to detect proprioceptive data at the foot. The computing device is in data communication with the sensor. The computing device is configured to receive the proprioceptive data detected by the sensor and to dynamically determine auditory cue instructions based on the proprioceptive data received from the sensor. The auditory device is controllably coupled to the computing device. The auditory device is configured to receive the auditory cue instructions from the computing device and to dynamically generate audible cues in response to the audible cue instructions received from the computing device.

### CROSS REFERENCE TO RELATED APPLICATIONS

This application claims priority to copending U.S. Application, Ser. No. 63/538,714, filed on Sep. 15, 2023, which is hereby incorporated by reference for all purposes.

### BACKGROUND

The present disclosure relates generally to medical systems. In particular, balance and gait assistance systems for people with peripheral neuropathy are described.

Maintaining balance is essential for human mobility and stability. Human balance relies on three primary systems: the vision system, the vestibular system in the inner ear, and the proprioceptive system in muscles and joints.

The proprioceptive system provides a sense of body awareness independent of visual cues. Body awareness enables maintaining one's balance in the dark when visual information is limited. The proprioceptive system detects force and pressure information at various muscle and joint locations, such as conducting nerve signals from the feet to the brain.

Peripheral neuropathy is a condition that affects the nerves outside the brain and spinal cord. Difficulty maintaining balance is a common condition for those with peripheral neuropathy. As a result of difficulties maintaining balance, people with peripheral neuropathy can find it difficult or unsafe to walk or stand without assistance.

Attempts to address balance difficulties resulting from peripheral neuropathy have been attempted, but there is not yet an ideal solution. Some attempts require a user to wear custom socks or shoes with sensors and actuators that provide feedback to a user's lower leg or foot. Conventional approaches depend on a threshold level of nerve capability in the user's feet and legs, which is not present for certain people with peripheral neuropathy. Conventional systems also do not utilize auditory cues, which could significantly enhance one's balance and gait.

Shoes and insoles to detect and record gait information exist for enhancing athletic performance. For example, some shoes with sensors are used to monitor and access the efficiency of running strides in elite athletes. While useful for optimizing athletic performance, conventional systems of shoes and insoles with sensors are not adapted to assist those with peripheral neuropathy to maintain balance or walk more effectively and safely.

Thus, there exists a need for improved balance and gait assistance systems. Examples of new and useful balance and gait assistance systems relevant to the needs existing in the field are discussed below.

Examples of references relevant to balance and gait assistance systems include U.S. Patent Publication No. 20060016255 and journal article Horsak, B., Dlapka, R., Iber, M. et al. SONIGait: a wireless instrumented insole device for real-time sonification of gait. J Multimodal User Interfaces 10, 195-206 (2016). The complete disclosures of the listed documents are incorporated herein by reference for all purposes.

### SUMMARY

The present disclosure is directed to balance and gait assistance systems including a sensor, a computing device, and an auditory device. The sensor is disposed proximate a foot of a user and configured to detect proprioceptive data at the foot.

The computing device is in data communication with the sensor. Further, the computing device is configured to receive the proprioceptive data detected by the sensor and to dynamically determine auditory cue instructions based on the proprioceptive data received from the sensor.

The auditory device is controllably coupled to the computing device. The auditory device is configured to receive the auditory cue instructions from the computing device and to dynamically generate audible cues in response to the audible cue instructions received from the computing device.

### BRIEF DESCRIPTION OF THE DRAWINGS

FIG. 1 is a schematic view of a balance and gait assistance system.

FIG. 2 is a perspective view of the balance and gait assistance system shown in FIG. 1 assisting a person walking.

FIG. 3 is a flow diagram of computer executable instructions utilized by a computing device of the balance and gait assistance system shown in FIG. 1.

FIG. 4 is a side elevation view of multiple proprioceptive sensors disposed in an insole of a shoe beneath a foot.

FIG. 5 is a front elevation view of the balance and gait assistance system shown in FIG. 1 assisting a user starting to lean to the left to maintain his balance by increasing the volume of audible cues produced by an auditory device proximate a left ear of the user.

### DETAILED DESCRIPTION

The disclosed balance and gait assistance systems will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various balance and gait assistance systems are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Communicatively coupled” means that an electronic device exchanges information with another electronic device, either wirelessly or with a wire-based connector, whether directly or indirectly through a communication network.

“Controllably coupled” means that an electronic device controls operation of another electronic device.

### Balance and Gait Assistance Systems

With reference to the figures, balance and gait assistance systems will now be described. The systems discussed herein function to assist a user to maintain balance while standing and walking via audible cues. The novel systems described below find particular application assisting people with peripheral neuropathy. Helpfully, the novel systems assist people with peripheral neuropathy to maintain balance while walking and standing despite their proprioceptive systems not adequately detecting force and pressure information at key muscle and joint locations.

The novel balance and gait assistance systems have applicability beyond assisting people with peripheral neuropathy. Improving one's performance in athletic endeavors is another area where the novel systems can be used. For example, one may use the system to improve his or her balance and swing mechanics when hitting a golf ball.

Another application for the novel balance and gait systems described herein is assisting people utilizing prosthetic devices. For example, the novel systems may be used to assist people to initially learn how to use prosthetic devices. Further, the novel systems described below can assist people to maintain their balance more effectively while using prosthetic devices after learning how to use them initially.

The reader will appreciate from the figures and description below that the presently disclosed balance and gait assistance systems address many of the shortcomings of conventional systems. For example, the novel balance and gait assistance systems do not require the user to have a threshold level of nerve capability in the user's feet and legs, which may not be present for certain people with peripheral neuropathy. Advantageously, the novel systems discussed herein utilize auditory cues to significantly and unobtrusively enhance one's balance and gait. The novel systems utilize commonly available components, such as smartphones and headphones or hearing aids, to make the novel systems convenient and widely accessible.

### Balance and Gait Assistance System Embodiment One

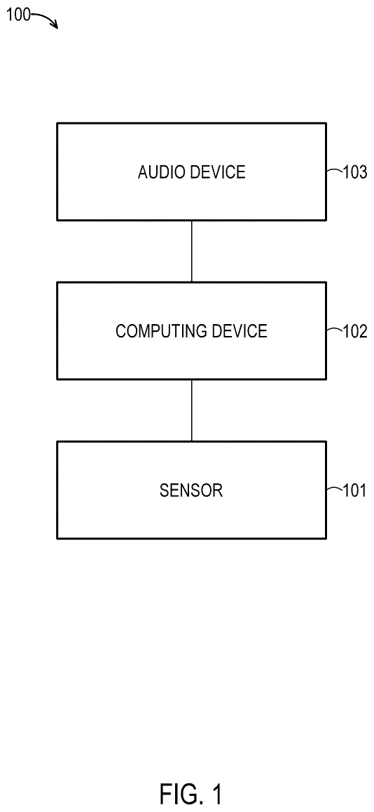

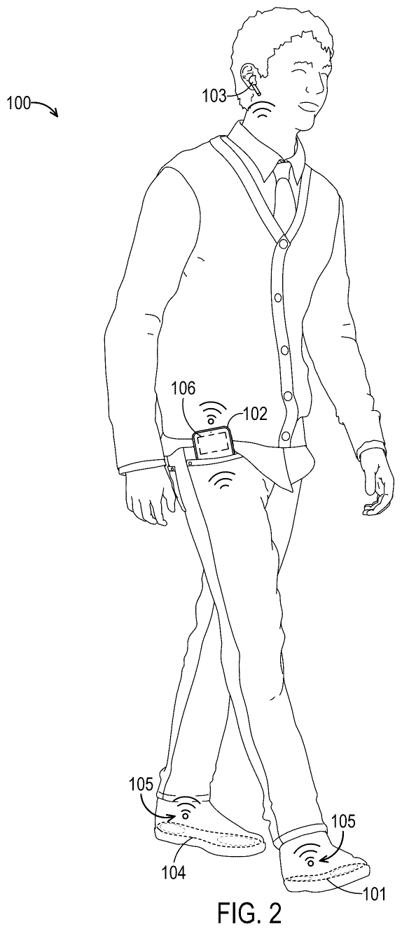

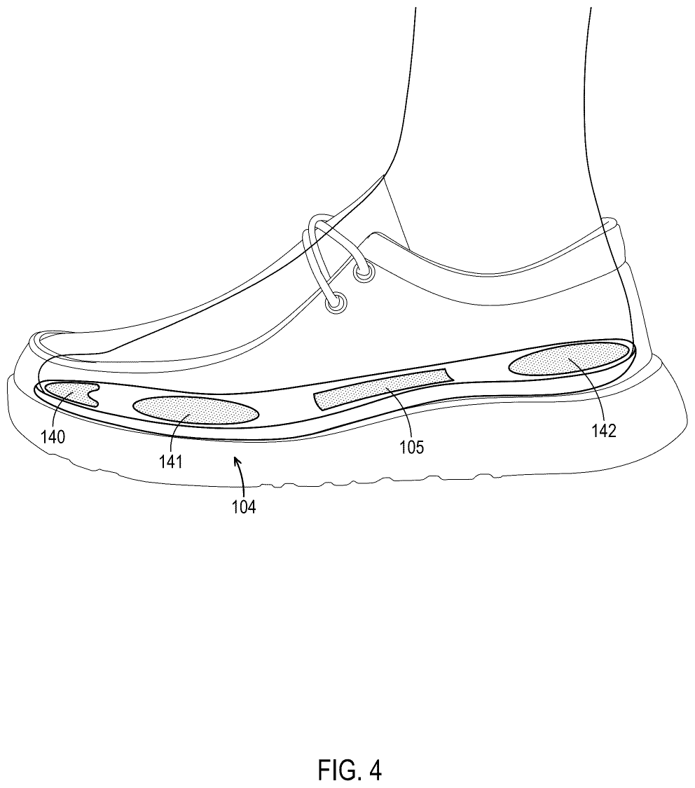

With reference to FIGS. 1-5, a first example of a balance and gait assistance system, balance and gait assistance system 100, will now be described. As shown in FIGS. 1 and 2, balance and gait assistance system 100 includes a first sensor 101, a second sensor 104, a computing device 102, and an audio device 103. The components of balance and gait assistance system 100 are described in the sections below.

In some examples, the system does not include one or more features included in system 100. For example, some system examples do not include a computing device, but instead include computer executable instructions for operating on a user supplied computing device. In other examples, the systems include additional or alternative features.

### Sensors

Sensors 101 and 104 function to detect proprioceptive data, including force and pressure information, at selected muscle or joint locations. The proprioceptive data detected by sensors 101 and 104 are utilized by computing device 102 to generate auditory balance cues reproduced by audio device 103.

The reader should understand that sensors 101 and 104 may be comprised of a plurality of sensors at a variety of muscle and joint locations. For example, sensor 104 includes an array of sensors 140, 141, and 142 incorporated into an insole to detect proprioceptive data at selected locations of a user's foot; namely, front, middle, and rear positions, respectively. While some sensor examples include a single sensor, many sensor examples include multiple sensors. The number of sensors may be selected to meet the needs of a give application.

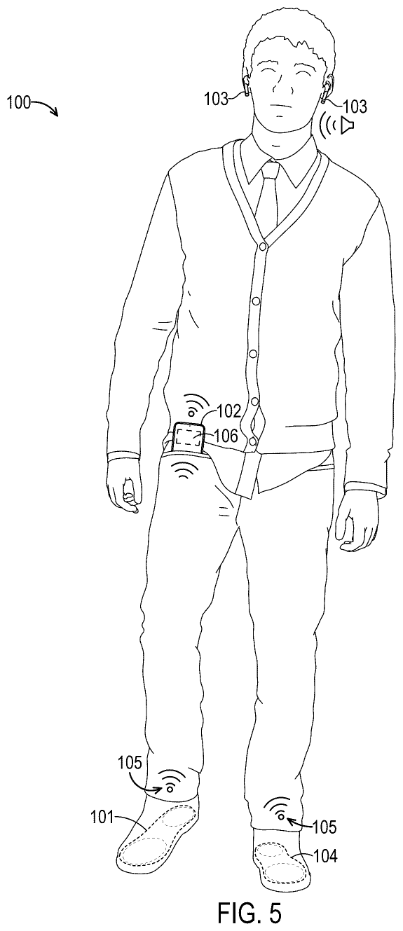

In the present example, sensors 101 and 104 are configured to detect proprioceptive data at the bottom of a user's foot. As shown in FIGS. 2 and 5, sensors 101 and 104 are disposed beneath the left and right feet of the user.

In various examples, the sensors are configured to detect proprioceptive data at additional or alternative locations than the bottom of a user's foot. For example, the sensor may be configured to detect proprioceptive data at the bottom, top, or sides of the foot; at the ankle; at the lower leg; at the knee; at the upper leg; and/or at the hip. The sensors may be located at any physiological location that provides useful proprioceptive data to help maintain balance while standing or walking.

In the present example, sensors 101 and 104 are incorporated into a removable insole. However, in other examples the sensors are not incorporated into an insole. For example, the sensors may be incorporated into a shoe, into a sock, into pants, and/or into a wrap. In some examples, the sensors are adhered to a selected location.

The sensors may be any currently known or later developed type of sensor suitable for detecting physiological force and pressure information or other proprioceptive data. In some examples, the sensors additionally capture movement and/or position data.

In certain examples, radar-based sensors are included in the balance and gait assistance systems. Suitable radar-based sensors include those commonly used in canes for assisting people with vision impairments. Radar proximity and terrain data may be used by the computing device along with proprioceptive data to generate audible cues.

In the example shown in FIGS. 2 and 5, sensors 101 and 104 include sensor transmitters 105 for communicating data wirelessly to computing device 102. In some examples, the sensors additionally or alternatively include data cables complementarily configured with the computing device to selectively couple with the computing device and exchange data with the computing device via the data cable. Thus, the sensors may commutatively couple to the computing device via wired or wireless means.

Sensor transmitters 105 in sensors 101 and 104 are Bluetooth™ transmitters utilizing the Bluetooth™ data protocol. However, any suitable wireless data transmission may be used, such as WiFi™ radio waves, Zigbee™, Z-Wave™, and NFC™ transmission protocols.

In the present example, sensor transmitters 105 in sensors 101 and 104 are configured to transmit data at 1 megabit per second or faster, which enables real-time proprioceptive data to be processed by computing device 102. Data transmission protocols enabling real-time or substantially real time data exchange are preferred. Speedy data transmission facilitates proprioceptive data from the sensor being processed by the computing device and translated into audio balance cues reproduced by the audio device substantially simultaneously with the physiological event giving rise to the proprioceptive data.

In some examples, one or more of the sensors further includes a wireless data receiver configured to receive data, including instruction data, from the computing device. For example, the sensor may receive instructions from the computing device to increase or decrease sensitivity, polling frequency, or other sensor attributes. Thus, the sensor may be controllably coupled to the computing device in addition to communicatively coupled.

### Computing Device

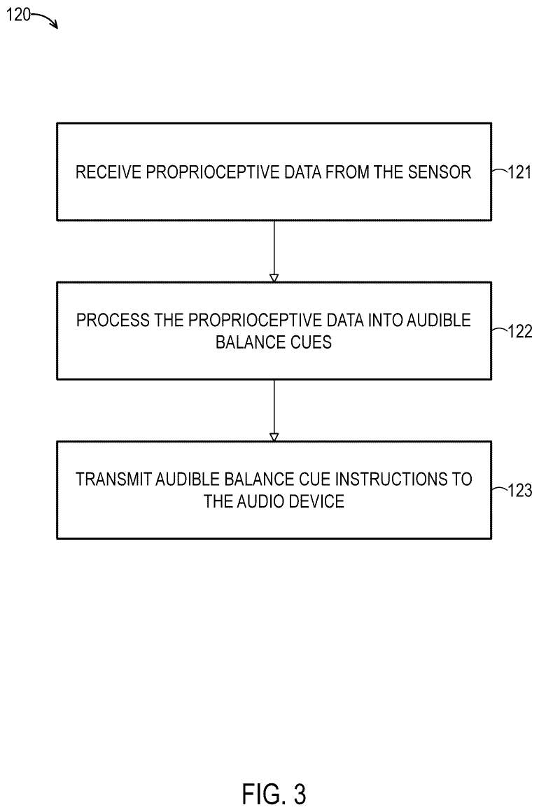

Computing device 102 functions to convert proprioceptive data received from sensors 101 and 104 into audible balance cues for audio device 103 to produce. Computing device 102 is configured to receive the proprioceptive data detected by sensors 101 and 104 and to dynamically determine auditory cue instructions based on the proprioceptive data. In some examples, the computing device receives data about the terrain a person is currently walking on, such as from a radar sensor in a cane, and factors the terrain data into the audible balance cues generated. As shown in FIG. 3, computing device 102 operates pursuant to computer executable instructions 120.

In the present example, computing device 102 is a standalone computing device physically separate from audio device 103. However, in some examples the computing device is integrated with the audio device. For example, the audio device may be a hearing aid or other device including a processor configured to execute computer instructions stored in local memory and the computing device may comprise the same processor and local memory.

In the example shown in FIGS. 2 and 5, computing device 102 is a smartphone. However, the computing device may be any currently known or later developed type of computing device. Suitable computing devices include smartphones, smart watches, smart rings, tablet computers, laptop computers, desktop computers, personal data assistants, media players, and ebooks. Portable computing devices are preferred to facilitate balance assistance wherever one using the system desires to go, but may be a fixed location computing device as well.

As shown in FIGS. 2 and 5, computing device 102 may be stored in a pocket and used for the balance and gait system without active involvement of the user. In some instances, the computing device may include a strap, lanyard, holster clip, or hook-and-loop fastener to secure the computing device to the person. In smartwatch or smart ring embodiments, the computing device may secure to the users wrist or finger directly.

Generally a single computing device is included in the system, but the system may include multiple computing devices. For example, the system may include a computing device for each foot or each body part monitored for proprioceptive data. The system may include a computing device for each sensor utilized and/or for each audio device utilized.

FIG. 3 demonstrates computer executable instructions that computing device 102 uses to generate audible balance cues based on proprioceptive data from sensors 101 and 104. Step 121 entails receiving proprioceptive data from one or more of sensors 101 and 104. At step 122, computing device 102 processes the proprioceptive data into audible balance cues. At step 123, computing device 102 transmits audible balance cue instructions to audio device 103.

Processing the proprioceptive data at step 122 may include referencing data tables correlating proprioceptive data with balance assistance parameter data. Step 122 may further include correlating balance assistance parameter data with audible cue parameter data. A further step may entail correlating audible cue data with audible cue instruction data. The correlations may be performed by data table lookups. The data tables enabling the correlations may be stored in local memory or may be stored in memory at remote locations accessible via a data network.

In the present example, the audible cue instructions include volume instructions. The volume instructions cause auditory device 103 to vary the volume of the audible balance cues based on a magnitude of balance correction required as determined by computing device 102 from the proprioceptive data.

The computing device may include user interfaces for adjusting settings of the system and/or for reviewing data utilized by the system. The user interfaces may be any suitable form or type of user interface.

As shown in FIGS. 2 and 5, computing device 102 includes a computer transmitter 106. Computer transmitter 106 is configured to transmit the auditory cue instructions wirelessly to the auditory device. In other examples, the computing device communicates the auditory cue instructions via a data cable.

In the present example, computer transmitter 106 is configured to transmit data at a frequency of at least 1 megabits per second. Transmitting data at a sufficiently high rate to enables producing real-time auditory cues based on the proprioceptive data detected by the sensor at a given moment. Real-time auditory cues have been observed to more effectively assist a user maintain balance than auditory cues that are delivered with a perceptible delay. The computer transmitter may transmit data at various rates faster or slower than 1 megabit per second.

### Audio Device

Audio device 103 functions to produce audible balance cues in response to instructions from computing device 102. As shown in FIG. 2, audio device 103 is disposed near each of the user's ears, but it may be disposed anywhere the user can hear the audible balance cues produced.

The audible balance cues are designed to correlate with proprioceptive sensor data detected by sensors 101 and 104. In particular, the audible balance cues provide the user with physiological feedback and instructions to better maintain his or her balance. The audible balance cues are configured to reinforce proper foot contact progression of normal gait. Further, the audible balance cues may signal when the user's center of gravity is deviating from a position conducive to maintaining balance.

In preferred examples, the audible balance cues are subliminal or substantially subliminal. In other examples, the audible balance cues produce consciously apparent cues for the user to follow. In some instances, the audible balance cues are audio tones. In certain examples, the audible balance cues are verbal messages.

In some examples, the volume of the audible balance cues varies to signify the intensity of balance correction required. For example, the volume of the audible balance cues may be relatively low when the proprioceptive data indicates that the user is in a balance secure state. However, the volume may rapidly increase when the proprioceptive data indicates the user is at risk of losing his or her balance. The increased volume along with changes in the type of tone or message may serve as a warning to the user that he or she is at risk of losing balance.

In the present example, audio device 103 includes two earbuds commonly used for music and video audio and telephone conversation audio. However, the audio device may be any currently known or later developed type of audio device. Suitable audio devices include earbuds, headphones, hearing aids, and general purpose speakers.

The size, shape, and number of audio devices may vary in different examples. In some examples, the system includes a single audio device. In other examples, the system includes a separate audio device for each ear. In select examples, the system includes more than two audio devices.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A system for assisting with balance and gait, comprising:
a sensor disposed proximate a foot of a user and configured to detect proprioceptive data at the foot;
a computing device in data communication with the sensor, the computing device configured to receive the proprioceptive data detected by the sensor and to dynamically determine auditory cue instructions based on the proprioceptive data received from the sensor; and
an auditory device controllably coupled to the computing device, the auditory device configured to receive the auditory cue instructions from the computing device and to dynamically generate audible cues in response to the audible cue instructions received from the computing device.

2. The system of claim 1, wherein the proprioceptive data detected includes pressure exerted on the foot.

3. The system of claim 2, wherein the sensor is disposed beneath the foot.

4. The system of claim 3, wherein the sensor is incorporated into an insole of a shoe.

5. The system of claim 4, wherein:
the sensor defines a sensor array comprised of a plurality of sensor members each configured to detect proprioceptive data; and
the plurality of sensor members are operatively in data communication with the computing device.

6. The system of claim 5, wherein the computing device is configured to dynamically determine auditory cue instructions based on the proprioceptive data received from the plurality of sensor members.

7. The system of claim 1, wherein the sensor includes a sensor transmitter configured to transmit data wirelessly to the computing device.

8. The system of claim 7, wherein the sensor transmitter is configured to transmit data at a frequency of at least 1 megabits per second.

9. The system of claim 8, wherein the sensor transmitter utilizes Bluetooth data communication protocols.

10. The system of claim 9, wherein the computing device includes a computer transmitter configured to transmit the auditory cue instructions wirelessly to the auditory device.

11. The system of claim 10, wherein the computer transmitter is configured to transmit data at a frequency of at least 1 megabits per second to enable producing real-time auditory cues based on the proprioceptive data detected by the sensor at a given moment.

12. The system of claim 1, wherein:
the sensor defines a first sensor;
the foot defines a first foot;
the system further comprises a second sensor disposed proximate a second foot of the user and configured to detect proprioceptive data at the second foot.

13. The system of claim 12, wherein the computing device is in data communication with the second sensor.

14. The system of claim 13, wherein the computing device is further configured to dynamically determine auditory cue instructions based on the proprioceptive data received from the second sensor

15. The system of claim 1, wherein the auditory device is configured to produce auditory cues that are subliminal.

16. The system of claim 1, wherein the auditory cue instructions include volume instructions.

17. The system of claim 16, wherein the volume instructions cause the auditory device to increase the volume of the auditory cues when the proprioceptive data indicates that the user is at risk of losing his or her balance.

18. The system of claim 16, wherein the volume instructions cause the auditory device to vary the volume of the audible balance cues based on a magnitude of balance correction required determined by the computing device from the proprioceptive data.

19. A system for assisting with balance and gait, comprising:
computer executable instructions configured to be executed on a computing device, the computer executable instructions configured to generate auditory cue instructions based on proprioceptive data;
a sensor disposed proximate a foot of a user, the sensor configured to detect proprioceptive data at the foot and to communicate the proprioceptive data to a computing device executing the computer executable instructions; and
an auditory device configured to receive auditory cue instructions from a computing device executing the computer executable instructions and to dynamically generate audible cues in response to the audible cue instructions.

20. The system of claim 19, wherein:
the sensor and the auditory device are configured to communicate data wirelessly with a computing device executing the computer executable instructions; and
the computer executable instructions define a smartphone software application.
